(19) United States
(12) Patent Application Publication
(10) Pub. No.: US 2013/0079769 A1
(43) Pub. Date: Mar. 28, 2013

(54) ELECTROSURGICAL INSTRUMENT ASSEMBLY

(71) Applicants: Robert HUMBLE, Cardiff (GB); Bryan TISSINGTON, Cardiff (GB); Steven Lawrence IRELAND, Veldhoven (NL); David MORRIS, Rhondda (GB)

(72) Inventors: Robert HUMBLE, Cardiff (GB); Bryan TISSINGTON, Cardiff (GB); Steven Lawrence IRELAND, Veldhoven (NL); David MORRIS, Rhondda (GB)

(73) Assignee: GYRUS MEDICAL LIMITED, Cardiff (GB)

(21) Appl. No.: 13/607,071

(22) Filed: Sep. 7, 2012

(30) Foreign Application Priority Data
Sep. 26, 2011 (GB) 1116489.4

(51) Int. Cl. A61B 18/14

(52) U.S. Cl. CPC A61B 18/14
USPC 606/41; 29/592.1

(57) ABSTRACT

A method of assembling an electrosurgical instrument (1) includes the steps of forming components such as an elongate shaft (11), an electrode (2) and a base unit (3), presenting the electrode (2) to the base unit (3) such that the electrode (2) is temporarily located on the base unit (3), and applying a locking component (10), the locking component (10) holding the electrode (2) and the base unit (3) together. The locking component (10) is then secured to the elongate shaft (11) unit in such a way as to retain the electrode (2) on the base unit (3) and form the completed electrosurgical instrument. The locking component can be secured by means of a mechanical arrangement such as a bayonet fixing, or by a screw threaded connection.

CORRESPONDING APPLICATIONS

This application claims priority from United Kingdom patent application number 1116489.4, filed 26 Sep. 2011.

### TECHNICAL FIELD

This invention relates to a method of assembling an electrosurgical instrument, and also to an electrosurgical instrument suitable for the treatment of tissue. Such instruments are commonly used for the vaporisation and/or coagulation of tissue in surgical intervention, most commonly in “keyhole” or minimally invasive surgery, but also in “open” surgery.

### BACKGROUND TO THE INVENTION

Electrosurgical instruments are growing in sophistication and complexity, with the number of electrodes increasing and the size of electrodes decreasing. One of the major factors in electrosurgical instrument design is the difficulty and complexity of instrument assembly, as well as the increased risk of components breaking or becoming detached during use. There is a need for an improved way of assembling complex electrosurgical instruments, and the present invention attempts to address this problem.

### SUMMARY OF INVENTION

Accordingly, a method of assembling an electrosurgical instrument as provided, the method including:
firstly forming components including an elongate shaft, an electrode and an insulating base unit,
secondly presenting the electrode to the base unit such that the electrode is temporarily located on the base unit,
thirdly applying a locking component, the locking component holding the electrode and the base unit together, and
finally securing the locking component to the shaft to form the completed electrosurgical instrument.

Conventionally, the components for electrosurgical instruments are fabricated separately, and then assembled together to form the instrument. Each component is added sequentially and secured in place, normally by an adhesive such as an epoxy resin. In contrast, the present invention provides for an electrode to be temporarily located on the base unit (i.e. without permanent fixing such as by adhesive etc.) before being finally secured in position by a locking component which is then attached to the shaft of the electrosurgical instrument. The electrode is only secured to the base unit by the locking component, which is applied after the electrode has been placed in its desired position on the base unit.

In one convenient arrangement, the forming includes forming both first and second electrodes, and the presenting includes presenting both electrodes to the base unit so they are temporarily located on the base component separate one from another. This enables a bipolar electrosurgical instrument to be manufactured, with active and return electrodes separated by the insulator provided by the base unit. Other components, such as further insulating members or additional electrodes, can be added to the base unit before the locking component is applied.

Conveniently, applying the locking component comprises applying the locking component from the distal end of the base unit. In this way, the electrodes and other components are assembled on the base assembly, the locking component is applied from the distal end of the instrument, and then secured in place by whichever securing method as will now be described.

Preferably, securing the locking component to the shaft includes a mechanical interlock mechanism. According to one convenient arrangement, a first one of either the locking component or the elongate shaft includes a latch mechanism, designed to cooperate with a corresponding part of the second one of either the locking component or the elongate shaft, such that the securing the locking component to the shaft comprises latching the locking component and shaft one to the other. Conveniently, the latch mechanism comprises a bayonet assembly.

According to an alternative arrangement, the locking component and the elongate shaft include complementary screw-threaded portions, and the securing the locking component to the shaft comprises screwing the locking component on to the shaft.

### DESCRIPTION OF THE DRAWINGS

The invention will now be further described, by way of example only, with reference to the accompanying drawings, in which:

FIG. 1 is a schematic diagram of an electrosurgical instrument sub-assembly during a first stage of assembly in accordance with an embodiment of the present invention,

FIG. 2 is a schematic diagram of an electrosurgical instrument sub-assembly during a second stage of assembly in accordance with an embodiment of the present invention,

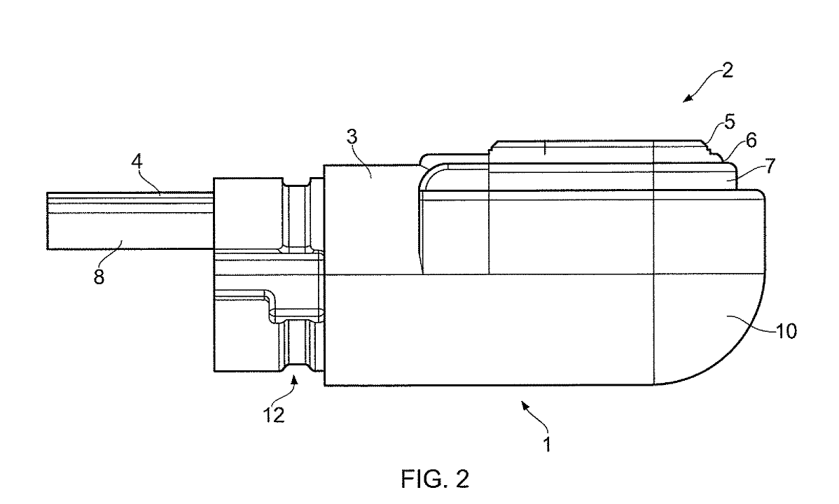

FIG. 3 is a schematic diagram of an electrosurgical instrument sub-assembly during a third stage of assembly in accordance with an embodiment of the present invention,

FIG. 4A is a schematic diagram of an electrosurgical instrument sub-assembly during a third stage of assembly in accordance with an alternative embodiment of the present invention,

FIG. 4B is a schematic diagram of the instrument sub-assembly of FIG. 4A, shown in a further connected stage of assembly.

FIG. 5 is a schematic exploded view of a part of an electrosurgical instrument during a first stage of assembly in accordance with another embodiment of the present invention,

FIG. 6 is a sectional side of the instrument of FIG. 5, shown in an assembled condition, and

FIG. 7 is a perspective view of the instrument of FIG. 6.

### DESCRIPTION OF EMBODIMENTS

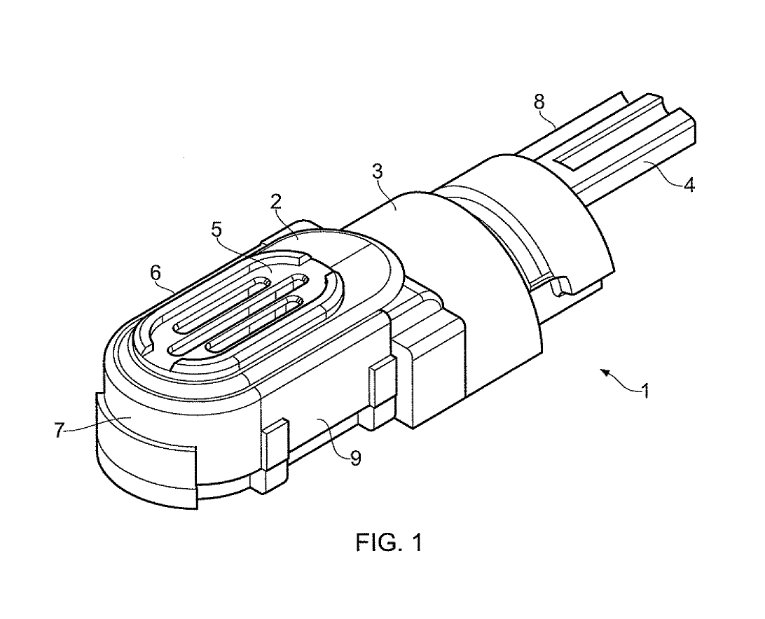

Referring to FIG. 1, the first stage of assembling an electrosurgical instrument shown generally at 1 comprises adding a metallic active electrode component 2 to a base unit in the form of a ceramic insulator component 3. The active electrode component 2 comprises an elongate tang 4 connected to a tissue contact portion 5. The component 2 is brought into engagement with the insulator component 3 from underneath the component 3, such that the tissue contact portion 5 is visible through an aperture 6 in the insulator component 3 with the tissue contact portion 5 abutting the edges of the aperture 6. The elongate tang 4 extends proximally from the insulator component 3, as shown in FIG. 1. The next stage in the assembly process is the addition of a metallic return electrode component 7, the component 7 being introduced from the distal end of the instrument 1 at the opposite end to the tang 4. The return electrode component comprises a tang 8 and a body portion 9, the component being introduced through an aperture (not shown) in the insulator component 3 such that the tang 8 lies alongside the tang 4 with the body portion 9 surrounding the tissue contact portion 5 of the active electrode component 2. This is the situation shown in FIG. 1.

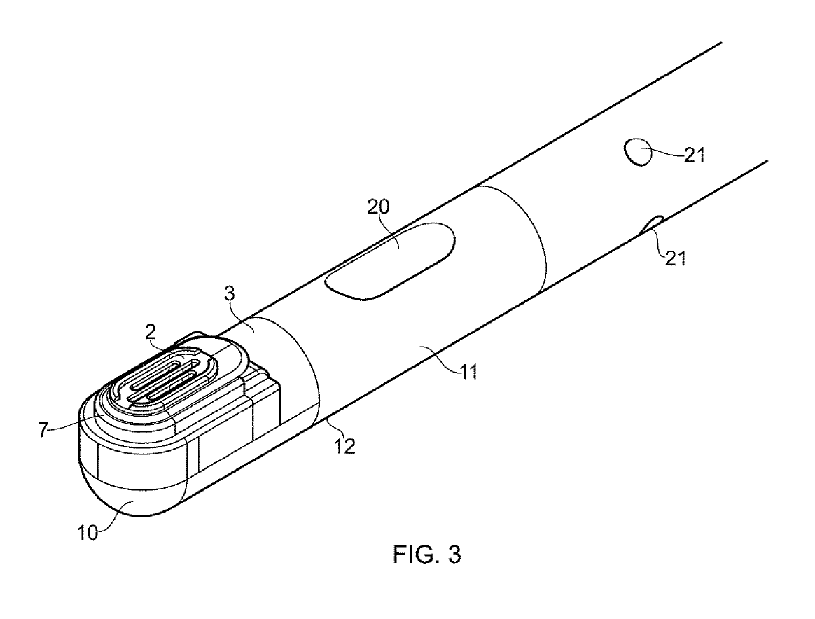

When the sub-assembly of components shown in FIG. 1 has been assembled, a locking component 10 is added to hold the components 2, 3, 7 & 8 in place. The locking component 10 is typically comprised of an insulating polymer or ceramic material, and is applied to the sub-assembly of FIG. 1 from the distal end of the instrument, as shown in FIG. 1 The locking component 10 is then attached to a tube component 11 forming the shaft of the electrosurgical instrument 1, the tube 11 having a bayonet assembly (not shown) adapted to connect with a corresponding bayonet assembly 12 provided on the locking component 10. An aperture 20 in the tube 11 allows access for the tang 4 to be connected to other components such as leads or a conductive suction tube (not shown). Once connected to the tube 11, the locking component 10 holds all of the other components in position one against another without the need for adhesive, although an adhesive may be optionally added to the components or a flowable material introduced through the aperture 20 in order to increase the strength of the interlocking assembly. Further apertures 21 are present in the proximal part of the tube 11 to allow for the introduction of further flowable material if desired. The tube 11 may be formed of a conductive material, or may contain a conductive band (not shown) if an additional return electrode is required. The assembled instrument is shown in FIG. 3.

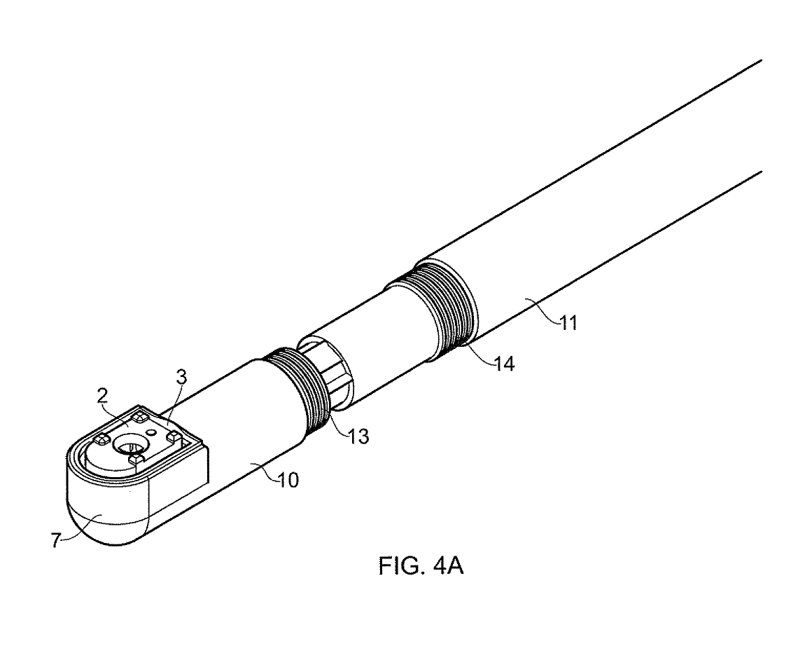

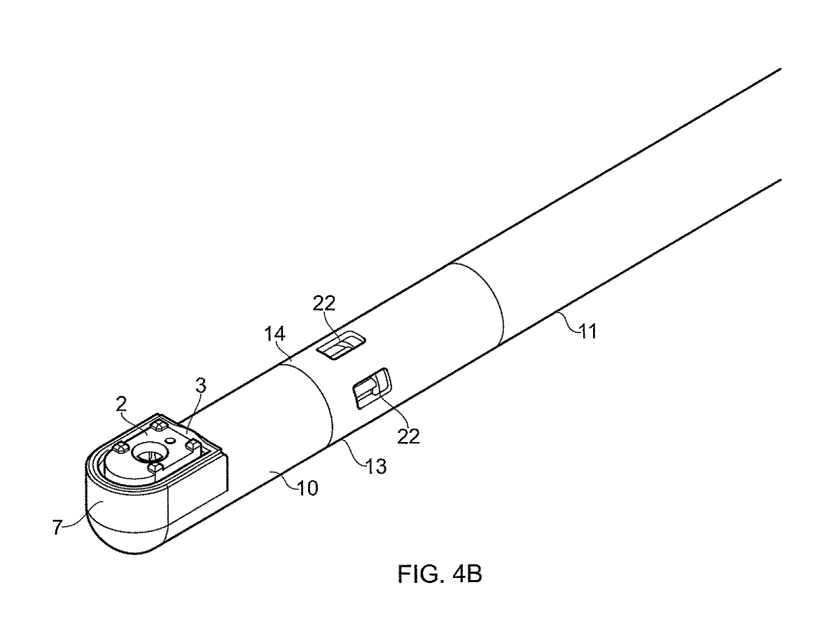

FIGS. 4A and 4B show a similar instrument 1 to that of FIG. 3, with similar components designated with similar reference numerals. The instrument of FIGS. 4A and 4B is different in that the active electrode component 2 is of a different design, and in that the locking component 10 is provided with a threaded portion 13, which connects with the tube 11 via a complementary threaded portion 14, When the tube 11 is added to the instrument and rotated to connect it to the locking component, the other components 2, 3, & 7 are all held firmly in place one against another with or without the application of additional adhesive material. Apertures 22 are present in the proximal part of the tube 11 to allow for the introduction of flowable material if desired. FIG. 4B shows the locking component 10 and tube 11 connected on to the other.

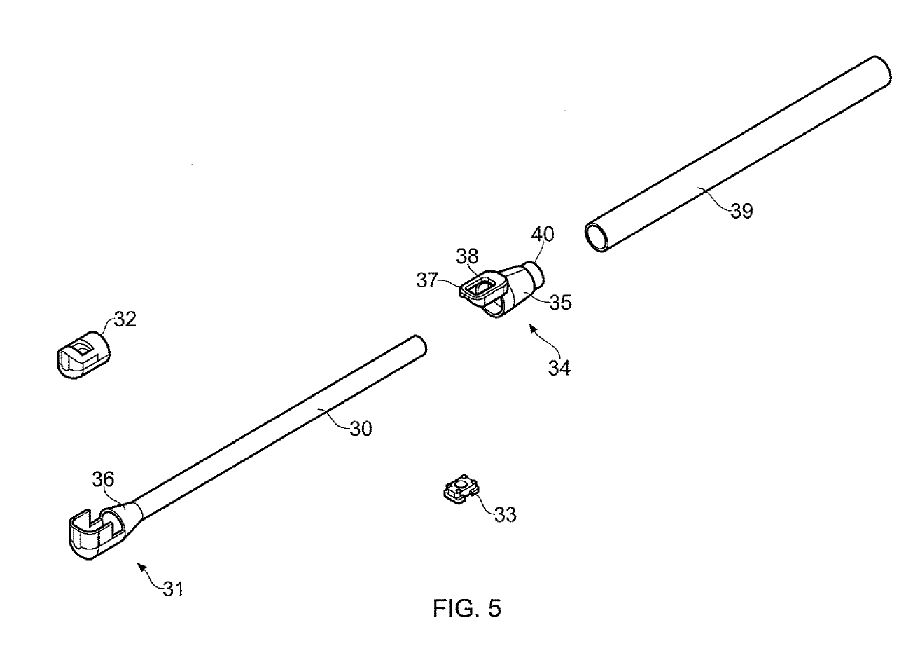

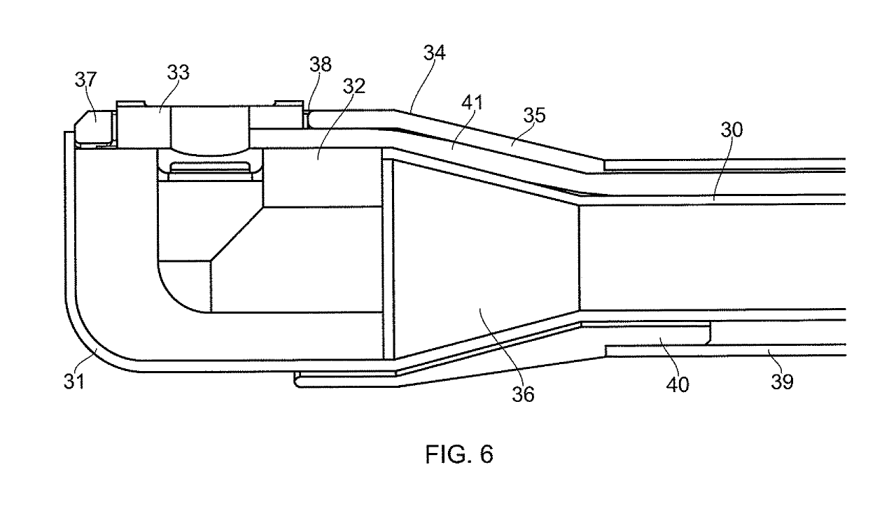

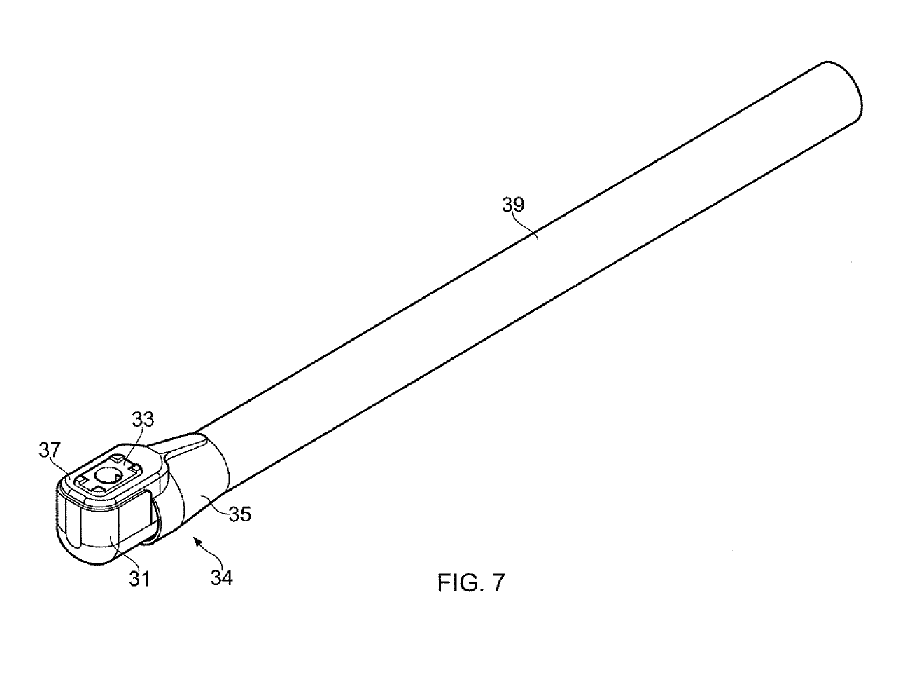

FIG. 5 shows an alternative embodiment of instrument, before assembly thereof. The components include a metallic tube 30 with a cradle 31 formed at its distal end. Into the cradle 31 is assembled a ceramic holder 32, on which is mounted an active electrode 33. Once the holder 32 and electrode 33 have been assembled on the cradle 31, an insulating locking member 34 is added, the locking member being introduced from the proximal end of the tube 30. The locking member comprises a conical body 35, adapted to cooperate with a corresponding conical section 36 present forming part of the cradle 31. The locking member 34 also includes a collar 37 having a central aperture 38 therein. When the locking member is introduced along the tube 30, it is affixed over the cradle 31 with its conical body 35 forming a tight press-fit connection with the conical section 36 of the cradle. The collar 37 fits over the ceramic holder 32, holding both it and the active electrode 33 in place, with the active electrode visible through the aperture 38 in the collar 37. The next stage of assembly is the addition of an insulating tube 39, secured over a corresponding circular lip 40 present on the proximal end of the locking member 34. Once assembled, as shown in FIGS. 6 and 7, the components can be secured in place by the introduction of a flowable material though apertures (not shown). A lead 41, shown in FIG. 6, connects the active electrode 33 to electrical connections at the proximal end of the instrument, and subsequently to an electrosurgical generator (not shown).

Other embodiments will be apparent to those skilled in the art without departing from the scope of the present invention. Other forms of locking component can be envisaged, as long as they can be used to hold together the other components that have been formed into a temporary sub-assembly ready to receive the locking assembly.

1. A method of assembling an electrosurgical instrument, the method including:
firstly forming components including an elongate shaft, an electrode and an insulating base unit,
secondly presenting the electrode to the base unit such that the electrode is temporarily located on the base unit,
thirdly applying a locking component, the locking component holding the electrode and the base unit together, and
finally securing the locking component to the shaft to form the completed electrosurgical instrument.

2. A method according to claim 1, wherein the forming includes forming both first and second electrodes, and presenting includes presenting both electrodes to the base unit so they are temporarily located on the base component separate one from another.

3. A method according to claim 1, wherein the applying the locking component comprises applying the locking component from the distal end of the base unit.

4. A method according to claim 1, wherein the securing the locking component to the shaft includes a mechanical interlock mechanism.

5. A method according to claim 4, wherein a first one of either the locking component or the elongate shaft includes a latch mechanism, designed to cooperate with a corresponding part of the second one of either the locking component or the elongate shaft, such that the securing the locking component to the shaft comprises latching the locking component and shaft one to the other.

6. A method according to claim 5, wherein the latch mechanism comprises a bayonet assembly.

7. A method according to claim 4, wherein the locking component and the elongate shaft include complementary screw-threaded portions, and the securing the locking component to the shaft comprises screwing the locking component on to the shaft.

8. An electrosurgical instrument, the electrosurgical instrument having been at least partially assembled by:
firstly forming components including an elongate shaft, an electrode and an insulating base unit,
secondly presenting the electrode to the base unit such that the electrode is temporarily located on the base unit,
thirdly applying a locking component, the locking component holding the electrode and the base unit together, and
finally securing the locking component to the shaft to form the completed electrosurgical instrument.

9. An electrosurgical instrument according to claim 8, wherein the forming includes forming both first and second electrodes, and the presenting includes presenting both electrodes to the base unit so they are temporarily located on the base component separate one from another.

10. An electrosurgical instrument according to claim 8, wherein the applying the locking component comprises applying the locking component from the distal end of the base unit.

11. An electrosurgical instrument according to claim 8, wherein the securing the locking component to the shaft includes a mechanical interlock mechanism.

12. An electrosurgical instrument according to claim 11, wherein a first one of either the locking component or the elongate shaft includes a latch mechanism, designed to cooperate with a corresponding part of the second one of either the locking component or the elongate shaft, such that the securing the locking component to the shaft comprises latching the locking component and shaft one to the other.

13. An electrosurgical instrument according to claim 12, wherein the latch mechanism comprises a bayonet assembly.

14. An electrosurgical instrument according to claim 11, wherein the locking component and the elongate shaft include complementary screw-threaded portions, and the securing the locking component to the shaft comprises screwing the locking component on to the shaft.

15. An electrosurgical instrument, comprising:
an elongate shaft,
a first electrode,
an insulating base unit, and
a locking component,
wherein the first electrode is held on the base unit by the locking component, and the locking component is secured to the elongate shaft to form the completed electrosurgical instrument.

16. An instrument according to claim 15, and further comprising a second electrode, wherein both the first and second electrodes are held on the base unit by the locking component separate from one another.

17. An instrument according to claim 15, wherein the locking component applies from the distal end of the base unit.

18. An instrument according to claim 15, wherein the locking component includes at least part of a mechanical interlock mechanism.

19. An instrument according to claim 18, wherein a first one of either the locking component or the elongate shaft includes a latch mechanism designed to cooperate with a corresponding part of the second one of either the locking component or the elongate shaft, such that the locking component is latched to the shaft, or vice versa.

20. An instrument according to claim 19, wherein the latch mechanism comprises a bayonet assembly.

21. A method according to claim 18, wherein the locking component and the elongate shaft include complementary screw-threaded portions, and the locking component is screwed on to the shaft, or vice versa.
